(19) United States
(12) Patent Application Publication
(10) Pub. No.: US 2006/0167524 A1
(43) Pub. Date: Jul. 27, 2006

(54) Current application apparatus and method for applying current

(75) Inventors: Teiyuu Kimura, Nagoya-city (JP); Minoru Makiguchi, Toyota-City (JP); Yoshiharu Yamamoto, Tokyo (JP)

Correspondence Address: POSZ LAW GROUP, PLC, 12040 SOUTH LAKES DRIVE, SUITE 101, RESTON, VA, 20191, US

(73) Assignees: Yoshiharu Yamamoto, Tokyo (JP); DENSO CORPORATION, Kariya-city (JP); TOYOTA JIDOSHA KABUSHIKI KAISHA, Toyota-city (JP)

(21) Appl. No.: 11/339,837

(22) Filed: Jan. 26, 2006

(30) Foreign Application Priority Data
Jan. 27, 2005 (JP) 2005-020176

(51) Int. Cl. A61N 1/18

(52) U.S. Cl. 607/45; 607/46

(57) ABSTRACT

A current application apparatus includes a pair of electrodes, mounts the pair of electrodes on the head by uses of an electrode mounting member, and applies a specific current to the head through the pair of electrodes. The pair of electrode is mounted by the electrode mounting member on one side of a lateral center of the head in such a manner that the following conditions are satisfied. One electrode of the pair is located on a skin located backward of an ear, which is positioned on the one side of the lateral center of the head, and the other electrode of the pair is located on a skin of the head located on the one side of the lateral center of the head. The specific current applied to the head has one of a white noise waveform and a 1/f noise waveform.

### CROSS REFERENCE TO RELATED APPLICATION

This application is based on and incorporates herein by reference Japanese Patent Applications No. 2005-020176 filed on Jan. 27, 2005.

### BACKGROUND OF THE INVENTION

1. Field of the Invention

This invention relates to a current application apparatus.

2. Description of Related Art

Conventionally, Japanese Unexamined Patent Publication No. H7-67970 and No. H8-150219 disclose treatment apparatuses, which treat stiffness and pain of people by applying an electrical stimulus to human bodies.

An effect to a center of a circulatory system affected by an stimulus to a vestibular system is described in “Yoshiharu Yamamoto et al.: Stochastic Resonance in Human Brain. Brain Science, 24: 591-594, 2002”. Inventors of the present application have further studied the effect, and as a result, the inventors discovered a phenomenon, where an electrical signal transmitted to a medulla oblongata is enhanced by a weak current from a vestibular nerve. This means that a stochastic resonance (SR) takes place in a human brain.

FIG. 9 shows a human nervous system. A brain stem shown in FIG. 9 includes a mesencephalon, pontes, a medulla oblongata, and many of autonomic nerves relating to life support are gathered in the brain stem. Nuclei of the medulla oblongata include a solitary nucleus and a nucleus of the vestibular nerve, and each of these nuclei are connected with each other. An afferent fiber called the vestibular nerve extends from the nucleus of the vestibular nerve, and the vestibular nerve is connected to an organ for equilibrium, such as three semicircular canals, in an inner ear. It becomes obvious by experiments that the electrical signal transmitted to the medulla oblongata is enhanced by the stochastic resonance, which is generated when the weak current is applied through the vestibular nerve. Then, an activity of the autonomic nerve can be activated when the enhanced electrical signal is transmitted to the medulla oblongata. However, any actual treatment apparatus that can be used in daily life has not been embodied yet.

### SUMMARY OF THE INVENTION

In view of the above disadvantages of the prior art, it is an objective of the present invention to provide a current application apparatus and a method for applying a current to obviate or mitigate at least one of the above disadvantages.

To achieve the objective of the present invention, there is provided a current application apparatus that is mountable on a head of a person, includes a pair of electrodes, mounts the pair of electrodes, and applies a specific current to the head. The current application apparatus includes an electrode mounting member to mount the pair of electrodes on the head. Here, the electrode mounting member is formed into such a shape that the electrode mounting member is supported by a part of the head. The pair of electrode is mounted by the electrode mounting member on one side of a lateral center of the head in such a manner that the following conditions are satisfied. One electrode of the pair is positioned on a skin located backward of an ear, which is positioned on the one side of the lateral center of the head, and the other electrode of the pair is positioned on a skin of the head located on the one side of the lateral center of the head. The current application apparatus applies the specific current to the head through the pair of the electrodes. Here, the specific current has one of a white noise waveform and a 1/f noise waveform.

To achieve the objective of the present invention, there is also provided a current application apparatus that is mountable on a head of a person, includes a first pair of electrodes and a second pair of electrodes, mounts the pair of electrodes on the head, and applies a specific current to the head. The current application apparatus includes an electrode mounting member to mount the first pair of electrodes and the second pair of electrodes on the head. The electrode mounting member is formed into such a shape that the electrode mounting member is supported by a part of the head. The first pair of electrodes and the second pair of electrodes are mounted on the head by the electrode mounting member in such a manner that the following conditions are satisfied. One electrode of the first pair is positioned on a skin located backward of an ear, which is positioned on a first side of a lateral center of the head. The other electrode of the first pair is positioned on a skin of the head located on a second side of the lateral center of the head, which is opposite from the first side of the lateral center of the head. One electrode of the second pair is positioned on a skin located backward of an ear, which is positioned on the second side of the lateral center of the head. The other electrode of the second pair is positioned on a skin of the head on the first side of the lateral center of the head. Then, the current application apparatus applies the specific current to the head through the first and second pairs of the electrodes. Here, the specific current has one of a white noise waveform and a 1/f noise waveform.

To achieve the objective of the present invention, there is also provided a method of applying a specific current to a head of a person. According to the method, a pair of electrodes on the head is mounted in such a manner that one electrode of the pair is positioned on a skin located backward of an ear, which is positioned on the one side of the lateral center of the head, and the other electrode of the pair is positioned on a skin of the head located on the one side of the lateral center of the head. Then, the specific current is applied to the head through the pair of the electrodes. Here, the specific current has one of a white noise waveform and a 1/f noise waveform.

### BRIEF DESCRIPTION OF THE DRAWINGS

The invention, together with additional objectives, features and advantages thereof, will be best understood from the following description, the appended claims and the accompanying drawings in which:

FIG. 1 is a block diagram showing a current application apparatus according to an embodiment of the present invention;

FIG. 2 is a diagram showing an arrangement of electrodes;

FIG. 3 is a diagram of an ear-mounting member;

FIG. 4 is an example of an eyeglass-type mounting member;

FIG. 5 is a diagram showing a noise waveform;

FIG. 6 is a diagram showing an effect corresponding to an output current strength;

FIG. 7 is a diagram showing another example of the eyeglass-type mounting member;

FIG. 8 is a diagram showing another example of the eyeglass-type mounting member; and

FIG. 9 is a diagram showing a structure of a human nervous system.

### DETAILED DESCRIPTION OF PREFERRED EMBODIMENT

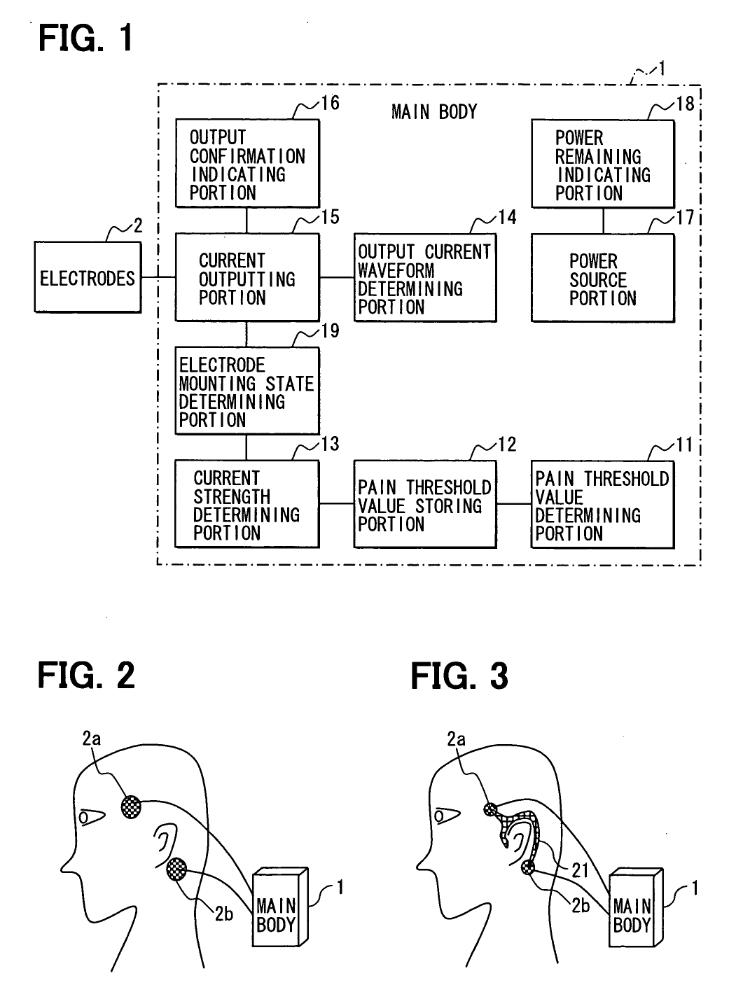

An embodiment of a current application apparatus according to the present invention will be described with reference to accompanying drawings. FIG. 1 is a block diagram showing the current application apparatus according to the present embodiment. As shown in FIG. 1, the current application apparatus includes a main body 1 and electrodes 2.

The electrodes 2 include a pair of electrodes 2a and 2b, and each electrode of the pair is mounted in position on a head of a person as shown in FIG. 2. One electrode of the pair is a positive electrode and the other is a negative electrode. A current applied by the main body 1 flows between the electrodes 2a and 2b. The pair of electrodes 2a and 2b is mounted on the head by use of an ear-mounting member 21 (see FIG. 3), which is formed into a shape that is supported by an ear. The ear-mounting member 21 is formed into an unobtrusive shape so that the present current application apparatus is easily used in daily life.

In the ear-mounting member 21, one electrode 2b of the pair is located on a skin located backward of an ear (e.g., a place around a bone called a “mastoid”), which is positioned on one side of a lateral center of the head, when the ear-mounting member 21 is mounted on the head. Also, the other electrode 2a of the pair is located on the skin of the head (e.g., a temple in FIG. 3) on the same side of the lateral center of the head as the one electrode 2b of the pair.

At the time of application of the current through the skin located backward of the ear, however, if the other electrode 2a of the pair is located on a skin on the other side of the lateral center of the head, which is opposite from the one side of the lateral center, where the one electrode 2b of the pair of electrodes is located, an equilibrium function of a vestibular system is affected. As a result, the person may be suffered from feeling dizzy.

Therefore, as shown in FIG. 2, in the present embodiment, the other electrode 2a of the pair of electrodes is located on the skin located on the same side of the lateral center of the head as the one electrode 2b of the pair so that the person does not feel dizzy when the person uses the current application apparatus. The other electrode 2a of the pair in FIG. 2 may be alternatively located on a forehead, and is not limited to be located on the temple.

As shown in FIG. 1, the main body 1 includes a pain threshold value determining portion 11, a pain threshold value storing portion 12, a current strength determining portion 13, an output current waveform determining portion 14, a current outputting portion 15, an output confirmation indicating portion 16, a power source portion 17, a power remaining indicating portion 18 and an electrode mounting state determining portion 19. A size of the main body 1 is a portable size such that the main body 1 is used in the daily life.

The pain threshold value determining portion 11 is, for instance, an input means, such as a push button. The pain threshold value determining portion 11 is operated when the person begins to sense application of an initial test current at the time of gradually increasing an amplitude of the initial test current applied through the electrodes 2 after the electrodes 2 are mounted on the head. The initial test current may have any kinds of waveforms, such as a white noise waveform, a 1/f noise waveform and a sine-wave waveform. In contrast, a specific current applied through the electrodes 2 is the current with a noise waveform that is one of the white noise waveform and the 1/f noise (i.e., pink noise) waveform. The noise waveform fluctuates around zero as shown in FIG. 5.

A weak current (i.e., the specific current), a current value of which is ranged between 50% and 100% level of a pain threshold current value (i.e., a threshold current value), is applied through a vestibular nerve so that stochastic resonance is effectively generated. This means that an electrical signal transmitted to a medulla oblongata is effectively enhanced. Here, the pain threshold current value needs to be set individually. Therefore, in order to individually set the pain threshold current value of the present current application apparatus, the pain threshold value determining portion 11 is operated when an individual person begins to sense the application of the initial test current at the timing of gradually increasing the amplitude of the noise waveform. The current value at the time of operating the pain threshold value determining portion 11 serves as the pain threshold current value.

The pain threshold value storing portion 12 stores the pain threshold current value, which is determined by the pain threshold value determining portion 11. The current strength determining portion 13 sets an output current strength between 50% and 100%. Here, the output current strength is a ratio of an output current value, at which the current is outputted through the electrodes 2, to the pain threshold current value stored in the pain threshold value storing portion 12.

The output current waveform determining portion 14 determines a waveform of an output current (i.e., the specific current) by selecting one of the white noise waveform and the 1/f noise waveform. In the white noise waveform, a frequency distribution of a power spectrum of the output current is uniform. In the 1/f noise waveform, the power spectrum of the output current is inversely proportional to a frequency of the output current.

As described above, it is necessary to apply the current with one of the white noise waveform and the 1/f noise waveform so that the stochastic resonance (SR) is effectively generated in the brain. According to the present current application apparatus, one of the two output current waveform (the white noise waveform, or the 1/f noise waveform) is selected, and the output current with the selected waveform is outputted at the output current value based on the pain threshold current value and the output current strength. Here, the output current strength is determined by the current strength determining portion 13 based on the selected waveform of the current. Therefore, the current with a specification determined by the output current waveform determining portion 14 is outputted through the electrodes 2.

It is noted that the output current waveform determining portion 14 may be constituted as an electrical circuit such that the output current waveform determining portion 14 may generate various waveforms described above. Also, the output current waveform determining portion 14 may read the above-described various waveforms stored in a memory to generate the waveforms.

Also, as shown in FIG. 6, it is known that when the white noise waveform is selected by the output current waveform determining portion 14, an effect of the stochastic resonance (SR) is maximized by setting the output current strength at about 80% and applying the output current to the head. Likewise, when the 1/f waveform is selected, the output current strength is set at about 60%. Therefore, the current strength determining portion 13 determines the output current strength correspondingly to the waveform, which is determined by the output current waveform determining portion 14.

The current outputting portion 15 outputs the current through the electrodes 2 with the specification determined based on the output current strength determining portion 13 and the output current waveform determining portion 14. Also, the current outputting portion 15 adjusts the output current value based on a determining result by the electrode mounting state determining portion 19, which will be described later.

The output confirmation indicating portion 16 includes an image generating portion (not illustrated) and a display screen (not illustrated). The output confirmation indicating portion 16 detects that the current with the specification determined by the output current strength determining portion 13 and the output current waveform determining portion 14 is applied to the head through the electrodes 2, and displays that the output is confirmed. Thus, the user can tell that the current is outputted from the electrodes 2. However, it is not so limited as discussed above. The output confirmation indicating portion 16 may announce that the current is outputted by use of a sound instead of the display.

The power source portion 17 supplies power to the main body 1, and uses a battery as a power source for generating the current. This allows the present current application apparatus easier to be used in the daily life. The power remaining indicating portion 18 includes an image generating portion (not illustrated) and a display screen (not illustrated), and detects a power voltage to display a battery remaining. This enables the user to understand the battery remaining.

When the current is outputted by the current outputting portion 15, the electrode mounting state determining portion 19 detects a resistance and a voltage level of the electrodes 2 to determine if the electrodes 2 contact the skin. When the electrode mounting state determining portion 19 determines that at least one of the electrodes 2 does not contact the skin, the current outputting portion 15 adjusts the output current value to be zero.

For instance, when the electrodes 2 are mounted again after the electrodes 2 are detached from the skin, a surge current may be applied through the electrodes 2. Thus, in the present current application apparatus, the output current value is adjusted to be zero when it is determined that at least one of the electrodes 2 does not contact the skin. Owning to this, the surge current is prohibited from being applied even when the electrodes are mounted again as described above.

Before using the present current application apparatus, firstly the electrodes are mounted on the head, secondly the power source to the main body 1 is activated, and lastly the operation for determining the pain threshold current value is performed. An ON/OFF switch may be provided in such a manner that the operation for determining the pain threshold current value is performed when the ON/OFF switch is ON, and the operation is omitted when the ON/OFF switch is OFF.

As discussed above, in the current application apparatus according to the present embodiment, the ear-mounting member 21 is used in such a manner that one electrode of the pair is located on the skin located backward of the ear on the one side of the lateral center of the head, and the other electrode of the pair is located on the skin at the temple located on the same side of the lateral center of the head as the one electrode of the pair. Also, the weak current having one of the white noise waveform and the 1/f noise waveform is applied through the pair of electrodes. Therefore, the current application apparatus can be used for recovery and improvement of an autonomic nerve activity in the daily life.

### First Modification of Embodiment

A first modification of the embodiment of the present invention will be described with reference to the accompanying drawings. Similar components of a current application apparatus of the first modification of the embodiment, which are similar to the components of the current application apparatus of the embodiment, will be indicated by the same numerals.

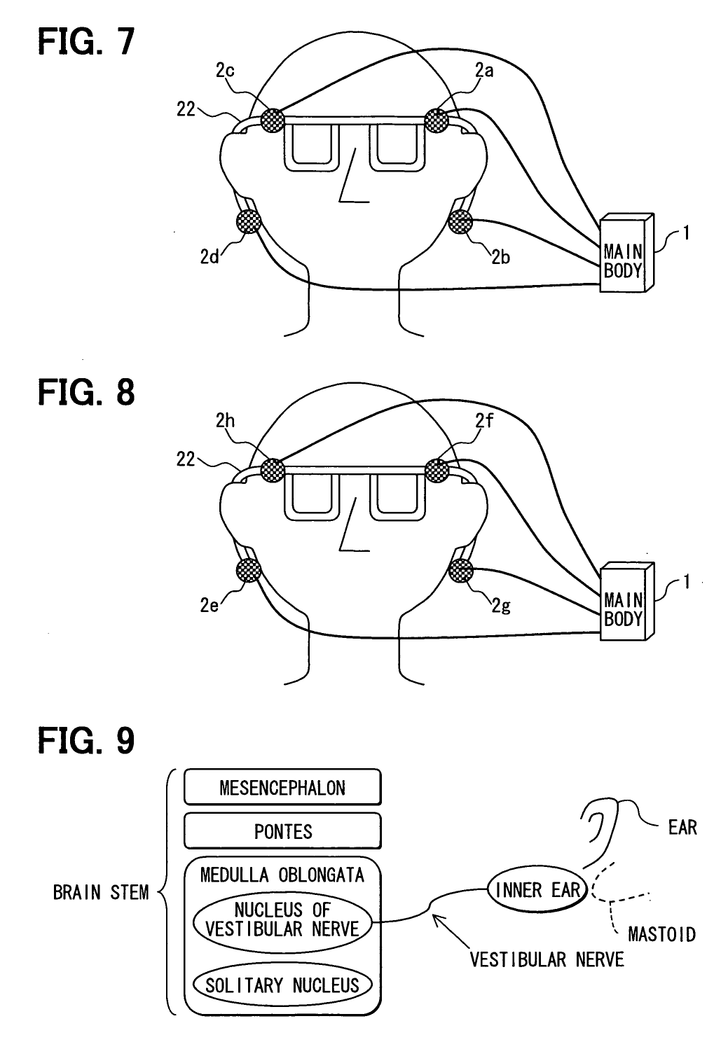

In the above-described embodiment, one pair of electrodes 2a and 2b is mounted on either of a right side of the head or a left side of the head (e.g., the left side in FIG. 2). However, the other pair of electrodes 2c and 2d may be mounted on the other side of the head, which is opposite side of the head from the one pair of the electrodes 2a and 2b, and the current is applied to each pair of electrodes 2a to 2d. This means that two pairs of electrodes 2a to 2d are mounted on the right side and the left side of the head correspondingly so that the current is applied through the two pair of electrodes (see FIG. 7). As a result, the current is bilaterally symmetrically applied so that the current is applied in a well-balanced manner.

A second modification of the embodiment of the present invention will be described with reference to the accompanying drawings. Similar components of a current application apparatus of the second modification of the embodiment, which are similar to the components of the current application apparatus of the embodiment, will be indicated by the same numerals.

In the above-described first modification of the embodiment, two pairs of electrodes are used, and one pair of electrodes 2a and 2b is located on one side of lateral center of the head, and the other pair of electrodes 2c and 2d is located on the other side of the lateral center of the head, which is opposite from the one pair of electrodes.

In the second modification of the embodiment, two pairs of electrodes 2e to 2h are used. However, in contrast, one electrode 2e of a first pair is located on the skin located backward of the ear on the right side of the lateral center of the head, and the other electrode 2f of the first pair is located on the skin of the head located on the left side of the lateral center of the head. Also, one electrode 2g of a second pair is located on the skin located backward of the ear on the left side of the lateral center of the head, and the other electrode 2h of the second pair is located on the skin of the head located on the right side of the lateral center of the head (see FIG. 8). Therefore, influences to the equilibrium function of the vestibular system are expected to be offset. As a result, the user may be limited from feeling dizzy.

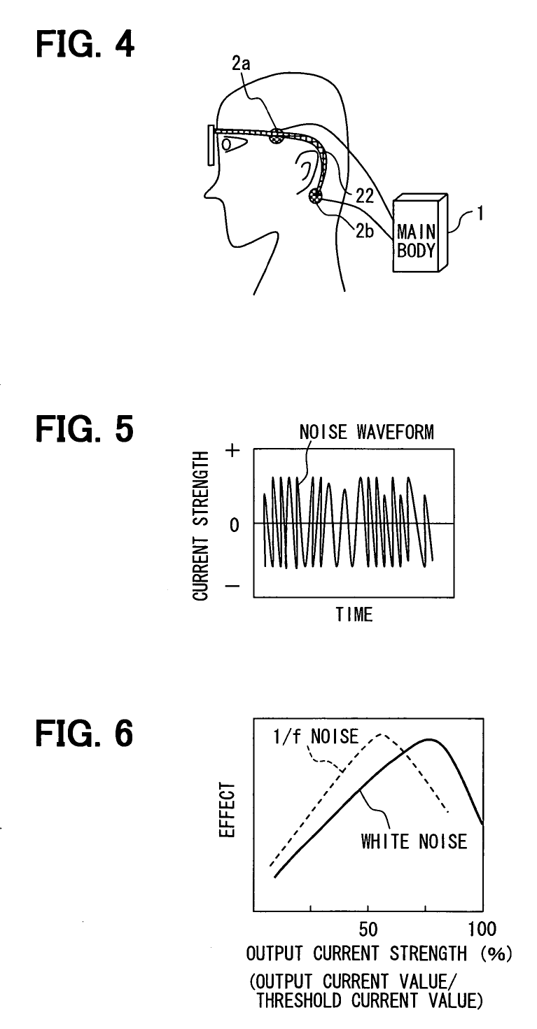

A third modification of the embodiment will be described. In the embodiment, the pair of electrodes 2a and 2b is mounted on the head by the ear-mounting member 21. However, the pair of electrodes 2a and 2b may be alternatively mounted on the head by an eyeglass-type mounting member 22 (see FIG. 4), which is supported by the ear and a nose. The eyeglass-type mounting member 22 is also formed into an unobtrusive shape so that the present current application apparatus is easily used in daily life.

Additional advantages and modifications will readily occur to those skilled in the art. The invention in its broader terms is therefore not limited to the specific details, representative apparatus, and illustrative examples shown and described. For instance, the pair of electrodes 2a and 2b shown in FIG. 2 may be attached to the skin by an adhesive material instead of the use of the ear-mounting member 21 or the eyeglass-type mounting member 22.

What is claimed is:

1. A current application apparatus mountable on a head of a person, comprising:
a pair of electrodes;
electrode mounting means for mounting the pair of electrodes on the head, wherein:
the electrode mounting means is formed into such a shape that the electrode mounting means is supported by a part of the head; and
the pair of electrode is mounted by the electrode mounting means on one side of a lateral center of the head in such a manner that the following conditions are satisfied:
one electrode of the pair is positioned on a skin located backward of an ear, which is positioned on the one side of the lateral center of the head; and
the other electrode of the pair is positioned on a skin of the head located on the one side of the lateral center of the head; and
current application means for applying a specific current to the head through the pair of the electrodes, wherein the specific current has one of a white noise waveform and a 1/f noise waveform.

2. The current application apparatus according to claim 1, wherein:
the pair of electrodes is a first pair of electrodes;
the one side of the lateral center of the head is a first side of the lateral center of the head;
the current application apparatus further comprises a second pair of electrodes;
the first pair of electrodes is mounted by the electrode mounting means on the first side of the lateral center of the head; and
the second pair of electrodes is mounted by the electrode mounting means on a second side of the lateral center of the head, which is opposite from the first side of the lateral center of the head.

3. The current application apparatus according to claim 1, wherein the part of the head, which supports the electrode mounting means, includes at least one of the ear and a nose of the person.

4. The current application apparatus according to claim 1, wherein the current application means includes:
threshold value determining means for determining a threshold current value, at which the person begins to sense application of an initial test current through the pair of electrodes when the initial test current is gradually increased;
threshold value storing means for storing the threshold current value, which is determined by the threshold value determining means;
output current strength determining means for determining an output current strength of the specific current, wherein the output current strength is a ratio of an output current value, at which the specific current is outputted to the head, to the threshold current value; and
current outputting means for outputting the specific current to the head of the person through the pair of electrodes at the output current value determined based on the threshold current value and the output current strength.

5. The current application apparatus according to claim 4, wherein the output current strength determining means sets the output current strength to be ranged between 50% and 100%.

6. The current application apparatus according to claim 4, wherein the output current strength determining means determines the output current strength of the specific current based on the one of the white noise waveform and the 1/f noise waveform.

7. The current application apparatus according to claim 4, further comprising current output informing means for informing that the specific current is outputted by the current outputting means.

8. The current application apparatus according to claim 4, further comprising:
electrode mounting state determining means for determining if both electrodes of the pair contact the skin of the head; and
output current adjusting means for adjusting the output current value based on a determining result of the electrode mounting state determining means.

9. The current application apparatus according to claim 8, wherein the output current adjusting means adjusts the output current value such that the output current value becomes zero level when the electrode mounting state determining means determines that at least one electrode of the pair does not contact the skin of the head.

10. The current application apparatus according to claim 1, wherein the current application means includes a battery as a power source of the current application apparatus.

11. The current application apparatus according to claim 10, further comprising battery remaining informing means for informing a battery remaining of the battery.

12. A current application apparatus mountable on a head of a person, comprising:
a first pair of electrodes;
a second pair of electrodes;
electrode mounting means for mounting the first pair of electrodes and the second pair of electrodes on the head, wherein:
the electrode mounting means is formed into such a shape that the electrode mounting means is supported by a part of the head; and
the first pair of electrodes and the second pair of electrodes are mounted on the head by the electrode mounting means in such a manner that the following conditions are satisfied:
one electrode of the first pair is positioned on a skin located backward of an ear, which is positioned on a first side of a lateral center of the head;
the other electrode of the first pair is positioned on a skin of the head located on a second side of the lateral center of the head, which is opposite from the first side of the lateral center of the head;
one electrode of the second pair is positioned on a skin located backward of an ear, which is positioned on the second side of the lateral center of the head; and
the other electrode of the second pair is positioned on a skin of the head located on the first side of the lateral center of the head; and
current application means for applying a specific current to the head through the first and second pairs of the electrodes, wherein the specific current has one of a white noise waveform and a 1/f noise waveform.

13. The current application apparatus according to claim 12, wherein the part of the head, which supports the electrode mounting means, includes at least one of the ear and a nose of the person.

14. The current application apparatus according to claim 12, wherein the current application means includes:
threshold value determining means for determining a threshold current value, at which the person begins to sense application of an initial test current through the pair of electrodes when the initial test current is gradually increased;
threshold value storing means for storing the threshold current value, which is determined by the threshold value determining means;
output current strength determining means for determining an output current strength of the specific current, wherein the output current strength is a ratio of an output current value, at which the specific current is outputted to the head, to the threshold current value; and
current outputting means for outputting the specific current to the head of the person through the pair of electrodes at the output current value determined based on the threshold current value and the output current strength.

15. The current application apparatus according to claim 14, wherein the output current strength determining means sets the output current strength to be ranged between 50% and 100%.

16. The current application apparatus according to claim 14, wherein the output current strength determining means determines the output current strength of the specific current based on the one of the white noise waveform and the 1/f noise waveform.

17. The current application apparatus according to claim 14, further comprising current output informing means for informing that the specific current is outputted by the current outputting means.

18. The current application apparatus according to claim 14, further comprising:
electrode mounting state determining means for determining if both electrodes of the pair contact the skin of the head; and
output current adjusting means for adjusting the output current value based on a determining result of the electrode mounting state determining means.

19. The current application apparatus according to claim 18, wherein the output current adjusting means adjusts the output current value such that the output current value becomes zero level when the electrode mounting state determining means determines that at least one electrode of the pair does not contact the skin of the head.

20. The current application apparatus according to claim 12, wherein the current application means includes a battery as a power source of the current application apparatus.

21. The current application apparatus according to claim 20, further comprising battery remaining informing means for informing a battery remaining of the battery.

22. A method of applying a specific current to a head of a person, comprising:
mounting a pair of electrodes on the head in such a manner that:
one electrode of the pair is positioned on a skin located backward of an ear, which is positioned on the one side of the lateral center of the head; and
the other electrode of the pair is positioned on a skin of the head located on the one side of the lateral center of the head; and
applying the specific current to the head through the pair of the electrodes, wherein the specific current has one of a white noise waveform and a 1/f noise waveform.

23. The method according to claim 22, further comprising:
gradually increasing a current value of an initial test current, which is outputted to the head, and determining a threshold current value, at which the person begins to sense application of the initial test current through the pair of electrodes;
storing the threshold current value, which is determined by the determining of the threshold current value; and
determining an output current value, at which the specific current is outputted to the head, to be less than or equal to the threshold current value that is stored by the storing of the threshold current value, wherein the applying of the specific current includes outputting the specific current to the head at the output current value determined by the determining of the output current value.

24. The method according to claim 23, wherein the output current value is ranged between 50% and 100% of the threshold current value.

25. The method according to claim 23, wherein the determining of the output current value includes determining the output current value based on the threshold current value and the one of the white noise waveform and the 1/f noise waveform.

26. The method according to claim 23, wherein:
the current output value is generally 80% of the threshold current value when the one of the white noise waveform and the 1/f noise waveform is the white noise waveform; and
the current output value is generally 60% of the threshold current value when the one of the white noise waveform and the 1/f noise waveform is the 1/f noise waveform.
